(19) United States
(12) Patent Application Publication
(10) Pub. No.: US 2024/0024292 A1
(43) Pub. Date: Jan. 25, 2024

(54) ATALUREN EYE DROP FORMULATION

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS, Paris (FR); SORBONNE UNIVERSITE, Paris (FR); UNIVERSITÉ PARIS CITÉ, Paris (FR)

(72) Inventors: Salvatore CISTERNINO, Paris (FR); Dominique BREMOND-GIGNAC, Paris (FR); Joël SCHLATTER, Paris (FR); Philippe-Henri SECRETAN, Paris (FR)

(21) Appl. No.: 18/257,372

(22) PCT Filed: Dec. 13, 2021
(86) PCT No.: PCT/EP2021/085413

§ 371 Date: Jun. 14, 2023

(30) Foreign Application Priority Data
Dec. 14, 2020 (EP) 20306557.8

(51) Int. Cl. A61K 31/4245; A61K 47/20; A61K 47/44; A61K 9/00; A61K 9/08

(52) U.S. Cl. CPC A61K 31/4245; A61K 47/20; A61K 47/44; A61K 9/0048; A61K 9/08

(57) ABSTRACT

Congenital aniridia is a rare and severe genetic panocular disease characterized by a complete or partial iris defect clinically detectable at birth. The most common form of aniridia occurring in around 90% cases is caused by PAX6 haploinsufficiency. Ataluren eye-drops aim to restore ocular surface PAX6 haploinsufficiency in Aniridia Related Keratopathy (ARK). However, they are currently no ophthalmic solution available forms. The objective of this study was to assess the physicochemical and microbiological stability of ataluren eye-drop 1% in preservative-free low-density polyethylene (LDPE) bottle with an innovative insert that maintains sterility after opening. Because ataluren is a strongly lipophilic compound, the formulation is complex and involves a strategy based on co-solvents in an aqueous phase or an oily formulation capable of totally dissolving the active ingredient. Throughout the 60 days period, the new formulation of the solution in LDPE bottle remained clear without any precipitation or color modification, no drug loss and no microbial development were detected. Thus, the present invention refers to a pharmaceutical solution comprising: ataluren or any one of its pharmaceutically acceptable derivatives, at least one solvent selected from the group consisting in dimethylsulfoxyde (DMSO), polyethylene glycol, polysorbate, glycerol, tyloxapol, and poloxamer, and castor oil; and its use for treating aniridia.

### FIELD OF THE INVENTION

The invention relates to pharmaceutical compositions comprising ataluren or any one of its derivatives and its use for treating congenital aniridia or limbal stem cell deficiency.

### BACKGROUND OF THE INVENTION

Congenital aniridia is a rare and severe ocular genetic disease. This panocular disease is characterized by a complete or partial iris defect clinically detectable at birth [1-3]. The disease is commonly associated with nystagmus, low vision, ptosis, corneal limbal insufficiency, glaucoma, cataract, optic nerve and foveal hypoplasia. Congenital aniridia affects equally males and females and has a prevalence of 1:40 000 to 1:100 000 [1]. The most common form of aniridia occurring in around 90% cases is caused by PAX6 haploinsufficiency, due to intragenic mutation or chromosomal rearrangement in the PAX6 gene at 11p13. An autosomal dominant transmission present in up to 90% of cases. Sporadic congenital aniridia may consist of 13% to 33% of cases as family forms consist in around two-third of cases. Congenital aniridia may be part of a syndrome as in WAGR contiguous gene syndrome (Wilms tumor, aniridia, genitourinary anomalies, and mental retardation) or in the rare Gillespie syndrome (cerebellar ataxia and mental retardation with ITPR1 mutation) [1-3]. A minority of different gene mutations may also be observed in congenital aniridia due other gene anomalies [1,4]. Visual prognosis of aniridia is severe with congenital low vision due to foveal hypoplasia and occasionally optic nerve hypoplasia. The severe evolution, results from corneal opacification, glaucoma, cataract, or keratopathy [1-6]. In the corneal limbus, the loss of stem cell niche in Vogt's palisades progresses and causes corneal opacity called aniridia-related keratopathy (ARK) [7]. Therefore, new approach for aniridia treatment has been proposed involving nonsense mutation suppression therapies such as ataluren that could limit aniridia disease progression and corneal damage [8-10]. As result of the study, a phase 2 clinical trial STAR was designed to evaluate the effect of oral ataluren in participants with nonsense mutation aniridia [11]. However, they are currently no ophthalmic solution available forms.

Ataluren, chemically known as 3-(5-(2-fluorophenyl)-1,2,4-oxadiazol-3-yl)benzoic acid, is a drug having nonsense codon suppression activity approved by the FDA and European agencies for the treatment of Duchenne muscular dystrophy in ambulatory patients aged from 2 years and older [12]. Ataluren enables ribosomal read-through of mRNA containing such as a premature stop codon, resulting in production of a full-length protein [13,14]. Ataluren is a small and lipophilic molecule soluble in organic solvents such as dimethyl sulfoxide (DMSO) and sparingly soluble in aqueous buffers (approximatively 0.5 mg/mL in 1:1 solution of DMSO: PBS pH 7.2) [15]. Currently, ataluren granules for oral suspension (Translarna® 125 mg, 250 mg, and 1000 mg, PTC Therapeutics International Limited, Dublin, Ireland) are the only marketed form in Europe. Preliminary stability study of extemporaneously ataluren ophthalmic suspension compounded in water vehicle using tween 80 (1%), hydroxypropylcellulose (1%), mixed in 0.9% sodium chloride showed a drug loss of at least 15% at 21 days and lack of complete ataluren dissolution. Other experiments were carried out by dissolving ataluren in pure castor oil and in a ready-to-use ophthalmic solution Cationorm® (Santen, France) prescribed as hydrating and lubricating eye drops. Despite the ophthalmic solution was limpid at the time of preparation precipitates were formed in less than 2 weeks. Alternative formulation strategies using DMSO as co-solvent was tested [16].

The repurposing of ataluren as an eye drop formulation in aniridia eye treatment could be advantageous to allow efficient corneal exposure and to limit systemic body exposure. Indeed, the formulation of an ataluren eye drops solution could be advantageous for the repositioning of ataluren in the ocular treatment of aniridia. Eye drops allow a more effective corneal exposure while limiting systemic body exposure. Ataluren is a small and lipophilic molecule soluble in organic solvents such as dimethylsufoxide (DMSO) and very slightly soluble in water [15]. Ataluren (1% m/v) suspension in 0.9% saline vehicle containing 1% tween 80 as a co-solvent, and 1% carboxymethylcellulose to increase viscosity, also known as the ‘START’ formulation, was shown to rescue the corneal deficit in Pax6-deficient mice model of aniridia [10]. Although this preclinical study suggests a benefit of the topical administration of ataluren, its chemical stability over time as well as its sterility were not assessed to our knowledge. Moreover, this ‘START’ formulation does not allow the dissolution of ataluren and it is thus not usable in humans. The objective of our study is to develop a 1% ataluren solution free of particles, chemically and microbiologically stable at least over 2 months when stored at 25±3° C. This new formulation could also be of interest for other genetic corneal limbal insufficiency. There is thus a need to identify novel eye drop formulations comprising ataluren in substantial amounts, and yet which remain suitable for ocular administration. There is also a need for ataluren eye drop formulations which remain easy to prepare, easy to administer, customizable, palatable, and with minimum potential adverse ingredients. There is also a need for ataluren eye drop formulations which allows the complete dissolution of ataluren and remains stable over time.

The invention has for purpose to meet the above-mentioned needs.

### SUMMARY OF THE INVENTION

Herein, the inventors formulate an ataluren oily solution free of water and particles at 1% and assess its chemical and microbiological stability in preservative-free formulation stored in low-density polyethylene (LDPE) opaque Novelia® bottles at ambient temperature (25±3° C. temperatures. Thus, the invention relates to a pharmaceutical solution comprising: i) ataluren or any one of its pharmaceutically acceptable derivatives; ii) at least one solvent selected from the group consisting in dimethylsulfoxyde (DMSO), polyethylene glycol, polysorbate (tween), glycerol, tyloxapol, poloxamer; and iii) castor oil.

In particular, the present invention is defined by the claims.

### DETAILED DESCRIPTION OF THE INVENTION

The inventors have assessed the physicochemical and microbiological stability of ataluren eye-drop 1% in preservative-free formulation stored in low-density polyethylene (LDPE) bottle with an innovative insert that maintains sterility after opening. Because ataluren is a strongly lipophilic compound and sensitive to the hydrolysis and oxidation, the formulation is complex and involves a strategy based on co-solvents in an aqueous phase or an oily formulation capable of totally dissolving the active ingredient. An oily formulation could protect better the compound from hydrolysis or oxidation degradation. The visual aspect, ataluren quantification by a stability-indicating chromatographic method, and microbiological sterility were analyzed. Throughout the 60 days period, the solution in LDPE bottle remained clear without any precipitation or color modification, no drug loss and no microbial development were detected. Thus, the inventors have demonstrated physical and microbiological stability of ataluren 1% eye-drop formulation at 25±3° C. which opens to further clinical studies and innovative treatment for patients.

Accordingly, the invention relates to a pharmaceutical solution comprising:
i) ataluren or any one of its pharmaceutically acceptable derivatives;
ii) at least one solvent selected from the group consisting in dimethylsulfoxyde (DMSO), polyethylene glycol, tween, glycerol, tyloxapol, poloxamer; and
iii) castor oil.

As used herein, the term “ataluren” also known as “Translarna” or “PTC124”, has its general meaning in the art and refers to the compound 3-(5-(2-fluorophenyl)-1,2,4-oxadiazol-3-yl)benzoic acid, which is of formula:

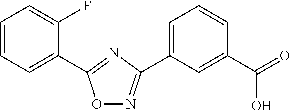 [SMILES: O=C(O)c1cccc(-c2noc(-c3ccccc3F)n2)c1]

Ataluren refers to a drug having nonsense codon suppression activity approved by the FDA and European agencies for the treatment of Duchenne muscular dystrophy in ambulatory patients aged from 2 years and older [12]. Ataluren enables ribosomal read-through of mRNA containing such as a premature stop codon, resulting in production of a full-length protein.

As used herein, the term “ataluren derivatives” has its general meaning in the art and refers to compounds derived from ataluren. Ataluren derivatives possess the desired pharmacological activity of ataluren, i.e. is capable to restore corneal transparency and ocular surface PAX6 haploinsufficiency in Aniridia Related Keratopathy.

In some embodiments, ataluren or any one of its pharmaceutically acceptable derivatives is contained in the pharmaceutical solution at a concentration ranging from 0.1 mg/mL to 15 mg/mL, more particular at a concentration ranging from 1 mg/mL to 15 mg/mL

In some embodiments, ataluren or any one of its pharmaceutically acceptable derivatives is contained in the pharmaceutical solution at a concentration of 0, 1, 0.5, 1, 2, 3, 4, 5, 6, 7, 8, 9, 10, 11, 12, 13, 14 or 15 mg/mL.

In some embodiments, ataluren or any one of its pharmaceutically acceptable derivatives is contained in the pharmaceutical solution at a concentration of 10 mg/mL.

In some embodiment, ataluren or any one of its pharmaceutically acceptable derivatives contained in the pharmaceutical solution is fully solubilized.

In some embodiment, the pharmaceutical solution comprises dimethylsulfoxyde (DMSO).

Thus, in some embodiment, the invention relates to a pharmaceutical solution comprising:
i) ataluren or any one of its pharmaceutically acceptable derivatives;
ii) dimethylsulfoxyde (DMSO); and
iii) castor oil.

In some embodiment, the pharmaceutical solution comprises 1, 2, 3, 4, 5 or 6 solvent selected from the group consisting in dimethylsulfoxyde (DMSO); polyethylene glycol; tween, glycerol; tyloxapol, poloxamer.

In some embodiment, the pharmaceutical solution comprises dimethylsulfoxyde (DMSO), polyethylene glycol; tween, glycerol; tyloxapol, and poloxamer.

As used herein, the term “dimethylsulfoxyde” (DMSO) has its general meaning in the art and refers to a highly polar organic reagent with the formula (CH₃)₂SO that has exceptional solvent properties for organic and inorganic chemicals. It is routinely used as solvent or in molecular biology, especially in the polymerase chain reaction (PCR), in transformation and transfection, for cell lysis, and in cytofluorimetric assessment.

As used herein, the term “polyethylene glycol” (PEG), also known as “polyethylene oxide” or “polyoxyethylene”, has its general meaning in the art and refers to a polyether compound with the formula C₂ₙH₄ₙ₊₂Oₙ₊₁. Polyethylene glycol shows characteristics of organic solvents. Polyethylene glycol includes but is not limited to PEG-300, PEG-400 or PEG-900.

As used herein, the term “tween”, also known as “polysorbate” has its general meaning in the art and refers to oily liquids derived from ethoxylated sorbitan esterified with fatty acids. Tween includes but is limited to tween 20 (also known as polysorbate 20, scattics or alkest TW tween 40, tween 60 and tween 80 (also known as polysorbate 80, montanox 80, or alkest TW 80). In particular embodiment, tween is tween 80.

As used herein, the term “glycerol”, also known as “glycerine”, has its general meaning in the art and refers to a polyol compound with the formula C₃H₈O₃. Glycerol is used as a solvent, humectant and vehicle in various pharmaceutical preparations.

As used herein, the term “tyloxapol” has its general meaning in the art and refers to a non-ionic liquid polymer of the alkyl aryl polyether alcohol type.

As used herein, the term “poloxamer” has its general meaning in the art and refers to nonionic triblock copolymers composed of a central hydrophobic chain of polyoxypropylene flanked by two hydrophilic chains of polyoxyethylene. Poloxamer includes but is not limited to poloxamer-407, poloxamer-184, poloxamer-188, poloxamer-124 or poloxomer-338.

As used herein, the term “castor oil” (DMSO) has its general meaning in the art and refers to a vegetable oil extracted from the seeds of the Ricinus communis plant, also known as castor beans. These seeds contain a toxic enzyme called ricin. However, the heating process that castor oil undergoes deactivates it, allowing the oil to be used safely. It's commonly used as an additive in foods, medications and skin care products, as well as an industrial lubricant and biodiesel fuel component. Indeed, castor oil exhibits most unusual physical and chemical properties due to the presence of ricinoleic acid in more than 87% quantities. The four functionalities, namely carboxylate, hydroxy, unsaturation, and long-chain hydrocarbon, present in ricinoleic acid made this molecule very unique in the chemical world.

Herein, the inventors demonstrated that the castor oil allows solubilization and also better protection of the ataluren (or any of its pharmaceutically derivatives) to oxidation.

In some embodiments, the pharmaceutical solution contains 50 to 99% of castor oil. In some embodiments, the pharmaceutical solution contains 50, 51, 52, 53, 54, 55, 56, 57, 58, 59, 61, 62, 63, 64, 65, 66, 67, 68, 69, 70, 71, 72, 73, 74, 75, 76, 78, 79, 80, 81, 82, 83, 84, 85, 86, 87, 88, 89, 90, 91, 92, 93, 94, 95, 96, 97, 98 or 99% of castor oil.

In some embodiments, the pharmaceutical solution contains 90% of castor oil.

As used herein, a pharmaceutical solution with 50 to 99% of castor oil refers to a pharmaceutical solution containing 50 to 99 volume of castor oil to 1 to 50 volume of solvent(s). Thus, a pharmaceutical solution with 90% of castor oil refers to a pharmaceutical solution containing 9 volume of castor oil to 1 volume of solvent(s).

As used herein, the term “pharmaceutical solution” refers to a formulation of a pharmaceutical active which renders the biological activity of the active ingredient (ataluren or anyone of its pharmaceutically acceptable derivatives) therapeutically effective, but which does not include other ingredients which are obviously toxic to a subject to which the formulation are intended to be administered.

As used herein, the term “pharmaceutically” or “pharmaceutically acceptable” refers to molecular entities and compositions that do not produce an adverse, allergic or other untoward reaction when administered to a mammal, especially a human, as appropriate.

In some embodiments, the pharmaceutical solution does not comprise water (water free pharmaceutical solution).

In some embodiments, the pharmaceutical solution may further comprise a preservative agent.

In some embodiments, the pharmaceutical solution does not comprise a preservative agent (preservative free pharmaceutical solution).

Examples of preservative agents which are suitable for the pharmaceutical solution may comprise sodium benzoate, benzoic acid, boric acid, sorbic acid and their salts thereof, benzyl alcohol, benzalkonium chloride, polidronium chloride (also known as Polyquad) parahydroxybenzoic acids and their alkyl esters, methyl and propyl parabens or their mixtures thereof.

In some embodiments, the pharmaceutical solution may further comprise an excipient, in particular one or more thickeners, such as a derivative of cellulose.

In some embodiments, the pharmaceutical solution may further comprise an antioxidant agent.

In some embodiments, the pharmaceutical solution does not comprise an antioxidant agent.

Examples of other antioxidant agents which are suitable for the pharmaceutical solution may comprise ascorbyl palmitate, butylated hydroxyanisole (BHA) and butylated hydroxytoluene (BHT), propyl gallate, vitamin E or their mixtures thereof.

In some embodiment, the pharmaceutical solution contains only:
i) ataluren or any one of its pharmaceutically acceptable derivatives;
ii) at least one solvent selected from the group consisting in dimethylsulfoxyde (DMSO), polyethylene glycol, tween, glycerol, tyloxapol, poloxamer; and
iii) castor oil.

In some embodiment, the pharmaceutical solution is a sterile solution.

As used herein, the term “sterile solution” refers to any form of administration which is substantially free, or even devoid of viable or revivable germs, potentially infectious, microbial known to those skilled in the art.

As used herein, the term “substantially free”, when used in relation to a given component of a solution (e.g. “a pharmaceutical solution substantially free of germs”), refers to a solution to which essentially none of said component has been added. When a solution is “substantially free” of a given component, said solution suitably comprises no more than 0.001 wt % of said component, suitably no more than 0.0001 wt % of said component, suitably no more than wt %, more suitably no more than 0.000001 wt.

As used herein, the term “solution” refers to a homogeneous liquid composition that generally does not contain solid particles, or that contains solid particles with an hydrodynamic radius inferior to 1 nm. In general, solutions can be distinguished from suspensions in that they cannot be separated by filtration. The term “solution” further includes solution stored and/or packaged in any recipient or container, sealed or not, which is suitable for pharmaceutical compositions, which may thus include any solution stored in vials, bottles such as ophthalmic bottle, intravenous (IV) bags, ampoules, cartridges and prefilled syringes. The solution of the present invention are solution compatible with ocular administration.

A solution compatible with ocular administration (i.e eye drop or ophthalmic solution) have to comply with wide range of guideline, such as defined in FDA guideline, EMA guideline or ASHP Guidelines on Pharmacy-Prepared Ophthalmic products (sterility, solubility, stability, viscosity, . . . ). For example, sterility is essential for ophthalmic solution, the solution being in direct contact to surface of the cornea and precorneal tissue.

Thus, the invention also relates to a container comprising the pharmaceutical solution of the present invention. Hence, the invention further relates to a container comprising a pharmaceutical solution, comprising:
i) ataluren or any one of its pharmaceutically acceptable derivatives;
ii) at least one solvent selected from the group consisting in dimethylsulfoxyde (DMSO); polyethylene glycol; tween, glycerol; tyloxapol, poloxamer; and
iii) castor oil.

In some embodiment, the pharmaceutical solution is preservative free.

As used herein, the term “container” refers to any primary or secondary packaging material which is compatible with the storage of the pharmaceutical solution. In a non-exhaustive manner, such container may include single-dose containers, multi-dose containers, well-closed containers, airtight containers, light-resistant containers. Such containers may be formed, completely or in-part, in glass, plastics, rubbers, paper/card boards and metals. For example, glass containers may include or consist of Type-I glass, Type-II glass, Type-III glass or any other non-parental usage glass. Plastic containers may include or consist of Urea formaldehyde (UF), Phenol formaldehyde, Melamine formaldehyde (MF), Epoxy resins (epoxides), Polyurethanes (PURs), Polyethylene, Polyvinylchloride, Polyethylene terephthalate (PET), Polyvinylidene chloride (PVdC), Polycarbonate Acrylonitrile butadiene styrene (ABS) or preservative-free low-density polyethylene (LDPE). Such containers may comprise or consist of vials, bottles, eyedropper bottle, intravenous (IV) bags, ampoules, cartridges and prefilled syringes.

In some embodiment, the container is a bottle.

In some embodiment, the container is an eye dropper bottle.

As used herein, the term “ophthalmic bottle”, also known as “eye dropper bottle” has its general meaning in the art and refers to container allowing as an ocular administration. Ophthalmic solutions should be directly administered to the eyes by patients themselves with controlling an eye dropper bottle.

In some embodiment, the container is a preservative-free low-density polyethylene (LDPE) bottle.

Ataluren eye-drops aim to restore ocular surface PAX6 haploinsufficiency in congenital aniridia in order to keep or recover corneal transparency. Herein, the inventors demonstrate that 1% ataluren eye-drop without preservative when stored at 22-25° C. in LDPE ophthalmic bottles remains stable during 60 days. Thus, this new formulation opens to further clinical studies and innovative treatment for patients.

Accordingly, in second aspect, the present invention refers to the pharmaceutical solution of the present invention for use as a medicament.

In particular, the present invention refers to a method for treating aniridia or limbal stem cell deficiency in a subject in need thereof comprising administering a therapeutically effective amount of the pharmaceutical solution of the present invention. In other words, the present invention refers to the pharmaceutical solution of the present invention for use in the treatment of aniridia in a subject in need thereof.

As used herein, the term “subject” refers to any mammals, such as a rodent, a feline, a canine, and a primate. Particularly, in the present invention, the subject is a human afflicted with or susceptible to be afflicted with congenital aniridia or limbal stem cell deficiency.

As used herein, the term “congenital aniridia” has its general meaning in the art and refers to a severe ocular disease characterized by a complete or partial iris defect. The disease is commonly associated with nystagmus, low vision, ptosis, corneal limbal insufficiency, glaucoma, cataract, optic nerve and foveal hypoplasia. Aniridia can be congenital or caused by a penetrant injury. Congenital aniridia is a panocular disease clinically detectable at birth [1-3]. The most common form of congenital aniridia occurring in around 90% cases is caused by PAX6 haploinsufficiency, due to intragenic mutation or chromosomal rearrangement in the PAX6 gene at 11p13. Sporadic congenital aniridia may consist of 13% to 33% of cases as family forms consist in around two-third of cases. Congenital aniridia in some individuals occurs as part of a syndrome, such as WAGR syndrome (kidney nephroblastoma (Wilms tumor), genitourinary anomalies and intellectual disability), or Gillespie syndrome (cerebellar ataxia).

As used herein, the term “limbal stem cell deficiency” (LSCD) has its general meaning in the art and refers to a loss or deficiency of the stem cells in the limbus that are vital for re-population of the corneal epithelium and to the barrier function of the limbus. This results in epithelial breakdown and persistent epithelial defects, corneal conjunctivalization and neovascularization, corneal scarring, and chronic inflammation. All of these contribute to loss of corneal clarity, potential vision loss, chronic pain, photophobia, and keratoplasty failure. LSCD has been associated with PAX6 gene mutations. Genetic disorders that have been reported with LSCD include peter's anomaly, ectrodactyly-ectodermal-dysplasia-clefting syndrome, keratitis-ichthyosis-deafness (KID) Syndrome, xeroderma pigmentosum, dominantly inherited keratitis, turner syndrome and dyskeratosis congenital.

As used herein, the term “treatment” or “treating” refer to prophylactic, palliative or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

As used herein, a “therapeutically effective amount” is intended for a minimal amount of active agent (i.e. ataluren in pharmaceutical solution) which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts.

As used herein the terms “administering” or “administration” refer to the act of injecting or otherwise physically delivering a substance as it exists outside the body (e.g. ataluren or any one of its pharmaceutically acceptable derivatives) into the subject, such as by ocular, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In some embodiment, the pharmaceutical solution of the present invention is administered by an ocular administration.

In a third aspect, the invention relates to a method for preparing the pharmaceutical solution as previously defined. Hence, the invention relates to a method for preparing the pharmaceutical solution of the invention comprising the steps of:
a) providing ataluren or any one of its pharmaceutically acceptable derivatives in powder form;
b) mixing said ataluren or pharmaceutically acceptable derivatives thereof in powder form, with at least one solvent selected from the group consisting in dimethylsulfoxyde (DMSO); polyethylene glycol; tween, glycerol; tyloxapol, poloxamer; and
c) adding castor oil and mixing the solution obtained, thereby preparing the pharmaceutical solution.

In some embodiment, the ataluren is providing at a concentration ranging from 0.1 to 15 mg/ml. In some embodiment, the ataluren is providing at a concentration of 0, 1, 0.5, 1, 2, 3, 4, 5, 6, 7, 8, 9, 10, 11, 12, 13, 14 or 15 mg/mL. In some embodiment, the ataluren is providing at a concentration of 10 mg/ml.

In some embodiment, ataluren is mixing at step b) in one volume of solvent(s), and nine volume of castor oil is added in step c).

In some embodiment, the method may further include the following step:
d) filtering the pharmaceutical solution obtained in step c), and
e) distributing the filtered pharmaceutical solution into a container.

As used herein, the term “filter” or filtering” has its general meaning in the art and refers to a process in which solid particulate matter is removed from a fluid, which can be either liquid or gas, using a porous medium for the process. Filtration can be easily applied to a large variety of pharmaceutical needs, and because of this flexibility it is widely used within the industry. The main filtration system are membrane filtration, depth filtration and cross-flow filtration. Membrane filtration is a physical separation method characterized by the ability to separate molecules of different sizes and characteristics. Its driving force is the difference in pressure between the two sides of a special filter membrane. Depth filtration use a porous filtration medium to retain particles throughout the medium, rather than just on the surface of the medium. It is primarily used for clarification of solutions. Among the most common filters used in depth filtration are ceramic-filtered and sintered filters such as pads, panels, thick cartridge, sand filter or lenticular. Cross-flow filtration is a filtration process in which feed water flows tangentially across a membrane surface. In cross-flow filtration a constant turbulent flow along the membrane surface prevents the accumulation of matter on the membrane surface. Filter membranes have different configurations. There are reverse osmosis (RO) membranes, ultrafiltration (UF) membranes, and nanofiltration (NF) membranes. Membranes are made of different types of materials such as cellulose acetate, cellulose nitrate, polyamide, polycarbonate, polypropylene, polytetrafluoroethylene and polysulfone.

In some embodiment, the pharmaceutical solution is filtered in step d) through a polyethersulfone filter, and in particular through a 0.22 μm polyethersulfone filter.

In some embodiment, the filtered pharmaceutical solution is distributed in step e) into an ophthalmic bottle.

The invention will be further illustrated by the following figures and examples. However, these examples and figures should not be interpreted in any way as limiting the scope of the present invention.

TABLE 1
Results from study of linearity. Slope: 2.0741 ± 0.0178. Y-
intercept: 10.2503 ± 1.2717. R2 > 0.9990

| Nominal concentration | Mean peak area ± SD | Calculated | Accuracy |
|---|---|---|---|
| (μg/mL) | (n = 45) | amount (μg/mL) | (%) |
| 50 | 114.44 ± 0.76 | 50.2 ± 0.7 | 100.5 |
| 60 | 134.05 ± 0.81 | 59.7 ± 0.5 | 99.5 |
| 70 | 155.69 ± 0.79 | 70.1 ± 0.6 | 100.2 |
| 80 | 175.69 ± 1.50 | 79.8 ± 1.2 | 99.7 |
| 90 | 197.32 ± 0.63 | 90.2 ± 0.6 | 100.2 |

TABLE 2
Relative standard deviation values (%) for repeatability
and intermediate precision (IQC).

| | Mean | | % RSD |
|---|---|---|---|
| | calculated | % RSD | intermediate |
| Theoretical | concentration | repeatability | precision |
| concentration (μg/mL) | (μg/mL) | (n = 6) | (n = 3) |
| 55 | 55.4 ± 0.6 | 0.57 | 0.79 |
| 70 | 70.5 ± 0.9 | 0.81 | 1.13 |
| 85 | 85.8 ± 1.8 | 1.46 | 2.02 |

TABLE 3
Forced degradation studies of 1% eye-drop oily formulation.

| | % Remaining (degradation) | | | | | |
|---|---|---|---|---|---|---|
| Stress conditions | Day 1 | | Day 3 | | Day 7 | |
| Acidic stress (0.1M HCl, 60° C.) | 100.1 | (+0.1) | 100.3 | (+0.3) | 100.4 | (+0.4) |
| Alkaline stress (1M NaOH, 60° C.) | 100.2 | (+0.2) | 99.9 | (−0.1) | 100.1 | (+0.1) |
| Oxidative stress (0.3% H₂O₂, 60° C.) | 100.2 | (+0.2) | 99.2 | (−0.8) | 98.1 | (−1.9) |
| Oxidative stress (15% H₂O₂, 60° C.) | 99.4 | (−0.6) | 89.7 | (−10.3) | 74.1 | (−25.9) |

TABLE 4
Direct photolysis of ataluren. The measurements corresponded
to a visible intensity of ~119,600 1x, and a UVA intensity
at 300-400 nm (66.5 W m⁻²).

| Time of exposition (min) | Mean peak area ± SD | % remaining |
|---|---|---|
| 30 | 240.2 ± 2.5 | 100.1 |
| 60 | 246.0 ± 1.9 | 102.4 |
| 180 | 240.4 ± 0.6 | 100.1 |
| 360 | 248.4 ± 2.1 | 103.4 |

TABLE 5
Chemical stability of ataluren 1% eye-drop oily formulation
stored at 22-25° C. in ophthalmic bottles and over time.

| | Actual | | | | | |
|---|---|---|---|---|---|---|
| | concentration | | | | | |
| | (100 mg/10 mL) | Mean ± SD % ataluren concentration remaining | | | | |
| Eyedropper | Day 0 | Day 5 | Day 15 | Day 21 | Day 30 | Day 60 |
| 1 | 100.7 ± 1.3 | 99.7 ± 1.3 | 99.2 ± 1.2 | 99.2 ± 1.3 | 100.8 ± 1.2 | 100.9 ± 1.4 |
| 2 | 100.4 ± 1.7 | 97.8 ± 1.2 | 99.4 ± 1.4 | 99.4 ± 1.5 | 101.5 ± 1.5 | 101.0 ± 1.9 |
| 3 | 100.3 ± 1.5 | 99.4 ± 1.3 | 98.6 ± 1.5 | 99.0 ± 1.1 | 100.1 ± 1.2 | 100.9 ± 1.5 |

TABLE 6
Chemical stability of the STAR ataluren suspension eyedrops (0.9% Sodium
chloride, 1% Tween 80, 1% Ataluren, 1% hydroxypropylcellulose) mixed in
0.9% sodium chloride, and stored at 25 ± 3° C. (n = 3 in triplicate).

| | Actual concentration | | |
|---|---|---|---|
| | (100 mg/10 mL) | Mean ± SD % ataluren concentration remaining | |
| Bottles | Day 0 | Day 7 | Day 21 |
| A | 100.4 ± 1.2 | 98.7 ± 1.1 | 84.0 ± 1.3 |
| B | 101.2 ± 1.5 | 97.9 ± 1.3 | 84.5 ± 1.4 |
| C | 99.8 ± 1.3 | 99.4 ± 1.1 | 84.0 ± 1.1 |

### FIGURES

FIG. 1: Reference chromatogram of ataluren 70 μg/mL in the oily solution.

FIG. 2. 95% accuracy profile for the dosage of ataluren in the oily solution by HPLC

FIG. 3. A. Chromatograms of ataluren obtained at day 0 (control), 1 day, 4 days and 7 days and its degradation products when exposed to 15% H2O2. B. Indirect photolysis of ataluren

### EXAMPLE

Material & Methods

Chemicals and Materials

Pharmaceutical ingredient of ataluren was obtained from Sigma-Aldrich (St. Quentin Fallavier, France). DMSO USP grade was provided from Wak-Chemie Medical GmbH (Cryosure, Steinbach, Germany). Pharmaceutical grade castor oil was provided from Cooper (Melun, France). Other chemicals were analytical grade. Titanium dioxide (99.5% Aeroxide® P25, nanopowder, average primary particle size 21 nm) came from Sigma Aldrich (St. Quentin Fallavier, France). All solvents used were HPLC grade from Merck (Darmstadt, Germany). Cationorm® was obtained from Santen (Evry, France) and contained mineral oils, cetalkonium chloride, tyloxapol, poloxamer 188, glycerin, buffer system (tris-HCl/trometamine) and water for injection. The sterile preservative free LDPE multidose eyedropper Novelia® was produced by Nemera (La Verpillere, France) and distributed by CAT laboratory (Montereau, France). It was chosen for its capacity to maintain sterility in normal use and under conditions of misuse and extended use including an anti-return valve system with a silicone membrane.

Formulation Development Assay and Preparation of Ataluren Eye-Drops

Preliminary stability study of extemporaneously ataluren ophthalmic suspension compounded in water vehicle using tween 80 (1%), hydroxypropylcellulose (1%), mixed in 0.9% sodium chloride showed a drug loss of at least 10% at 21 days and lack of complete ataluren dissolution. Other experiments were carried out by dissolving ataluren in pure castor oil and in a ready-to-use ophthalmic solution Cationorm® (Santen, France) prescribed as hydrating and lubricating eye drops. Despite the ophthalmic solution was limpid at the time of preparation precipitates were formed in less than 3 weeks. Alternative formulation strategies using DMSO as co-solvent was tested [16]. To completely solubilized ataluren, an optimized formulation using DMSO and castor oil was proposed. Eye-drop was compounded with 100 mg ataluren, 1 mL DMSO, and 9 mL castor oil. No preservative was added in the formulation. First, ataluren was mixed in DMSO until fully dissolved. Second, castor oil was added and the solution mixed by reversal during 1 min. The obtained ataluren solution was filtered through a 0.22 μm polyethersulfone filter (Millex, Merck Millipore, Fontenay-sous-Bois, France) and then sterilely distributed (10 mL per unit) into the eyedropper under the vertical laminar airflow hood of a B-class microbiological safety cabinet.

Analyses Performed on the Ataluren Solution

Visual Inspection

At each sample time, a visual inspection of eye-drop solution was made by the same operator, looking for a change in coloring, particles or precipitate, compared to a control consisting of castor oil.

Instrumentation

For each unit, ataluren was quantified using the liquid chromatography method adapted from the method described by Kong et al. [17]. Analyses were performed on a Thermo Scientific Ultimate 3000 chromatogram system (Villebon-sur-Yvette, France) including a quaternary pump (LPG 3400A), an automatic sampler (WPS 3000TSL), a diode array detector (DAD 300016) with 5 cm flow cell, and the associated software used to record and interpret chromatograms (Chromeleon®, Version 8.0). The stationary phase consisted in a Kinetex® C18 column (250×4.6 mm; 5 μm, Phenomenex, Le Pecq, France). The mobile phase was a gradient mixture of 0.1% formic acid (A) and acetonitrile (B). The flow rate was maintained at 1 mL/min, and the gradient profile was as follows: t0-11 min: A=30% B=70%; t11-15 min A=70% B=30%. The injection volume was 50 μL. The drug absorbance for quantification was obtained at 276 nm.

Method Validation

The HPLC method was validated for specificity, limit of detection (LOD), limit of quantification (LOQ), linearity, precision, accuracy, according to ICH Q2 validation guidelines [18]. Linearity was determined by preparing one calibration curve daily for three days using five concentrations of ataluren at 50, 60, 70, 80, and 90 μg/mL, diluted in acetonitrile. For each calibration, the slope, intercept, and regression coefficient (r) were calculated as regression parameters by the least square method. ANOVA tests were applied to determine applicability. The accuracy for the active compound was determined by analyzing three replicates of samples prepared at 80%, 100% and 120% of the target concentration. Accuracy was expressed as percentage of recovery determined by experimental concentration/theoretical concentration x 100. The acceptance criterion was ±2% deviation from the normal value for the recovery of ataluren. To verify the method precision, repeatability was estimated by calculating relative standard deviation (RSD) of intraday analysis and intermediate precision was evaluated using RSD of inter-day analysis. Both RSDs should be less than 2%. For that, each day for three days, six solutions of ataluren 1% were prepared, analyzed, and quantified using a calibration curve prepared the same day. The limit of detection (LOD) and limit of quantification (LOQ) for ataluren assay were determined by calibration curve method using the following equations:

$$LOD = \frac{3.3 \times SD of y - intercept}{Slope of calibration curve} \\ LOQ = \frac{10 \times SD of y - intercept}{Slope of calibration curve}$$

The matrix effect was evaluated by reproducing the previous methodology with the presence of all excipients present in the formulation and by comparing curves and intercepts.

The specificity was assessed by subjecting the ataluren 1% solubilized in DMSO (10%) and castor oil solutions to various forced degradation conditions: 0.1M hydrochloric acid (HCl), 0.1M sodium hydroxide (NaOH), 0.3% and 15% hydrogen peroxide (H₂O₂) for 24 h, 48 h, and 72 h at 60° C. To mimic the potential photodegradation occurring prior or after drug administration, photolytic studies were carried out by exposing the drug solutions to direct UV-visible light with and without photocatalyst or photosensitizer (i.e. titanium oxide 1 g L-1, riboflavin 100 mg L-1). Photolysis experiments were performed using a QSUN-XE-1 (Q-Lab, Bolton, United Kingdom) light chamber equipped with a xenon lamp, which simulates natural sunlight in a wavelength range of 300-800 nm. A Daylight-Q filter was used to simulate CIE Standard Illuminant D65 (Q-Lab) and irradiance was maintained constant (1.5 W m-2 at 420 nm). The measurements corresponded to a visible intensity of ˜119,600 1×, and a UVA intensity at 300-400 nm of 66.5 W m-2. For all the experiments the temperature was controlled and set at 25±0.5° C.

Sterility Assay

Sterility is an absolute requirement of ophthalmic formulations. To evaluate the sterility of the eye-drop in ophthalmic bottles, test for sterility carried out using the technique of membrane filtration with the product to be examined according to the European pharmacopeia [19]. To ensure applicability of the sterility test, sterility and fertility of media with and without the formulation were controlled. Six collection type strains were included corresponding to four bacteria (Pseudomonas aeruginosa ATCC 9027, Staphylococcus aureus ATCC 6538, Clostridium sporogenes ATCC 19404, and Bacillus subtilis ATCC 6633) and two fungi (Candida albicans ATCC 10231, Aspergillus brasiliensis ATCC 16404). Fluid thioglycollate medium and soya-bean casein digest medium were used as culture media. For each reference strain, 10 mL of formulations with and without the drug were filtered using the Steritest™ device (Steritest™ NEO, Merck Millipore). To validate the sterility applicability test, microbial growth clearly observable and visually comparable to that observed without product was observed each day during 5 days. To assess the sterility test of eye-drop ataluren oily solution, the same procedure was applied and the potential microbial growth was observed each day during 14 days.

Stability Study

Six bottles of the preparations were prepared, and bottles were stored at 25±3° C. Physical and chemical examinations were performed in triplicate immediately after preparation (Day 0) and at Day 1, 3, 7, 14, 30, 60, and 90 to define drug stability throughout its period of storage. The chemical stability of the extemporaneous preparation was defined by the drug content that contained not less than 90% and not more 110% of the labeled amount of ataluren [20].

Data Analysis-Acceptability Criteria

Data analyses were performed using Prism 6 (GraphPad Software, San Diego, USA). Descriptive statistics for continuous variables were expressed as mean±SD.

The study was conducted following methodological guidelines issued by the International Conference on Harmonisation (ICH) for stability studies [18]. The instability of ataluren solutions was considered by a variation of concentration outside the 90-110% range of initial concentration of drug and presence of degradation products. The observed solutions must be limpid, of unchanged color, and clear of visible signs of precipitation.

Results

Assay Validation

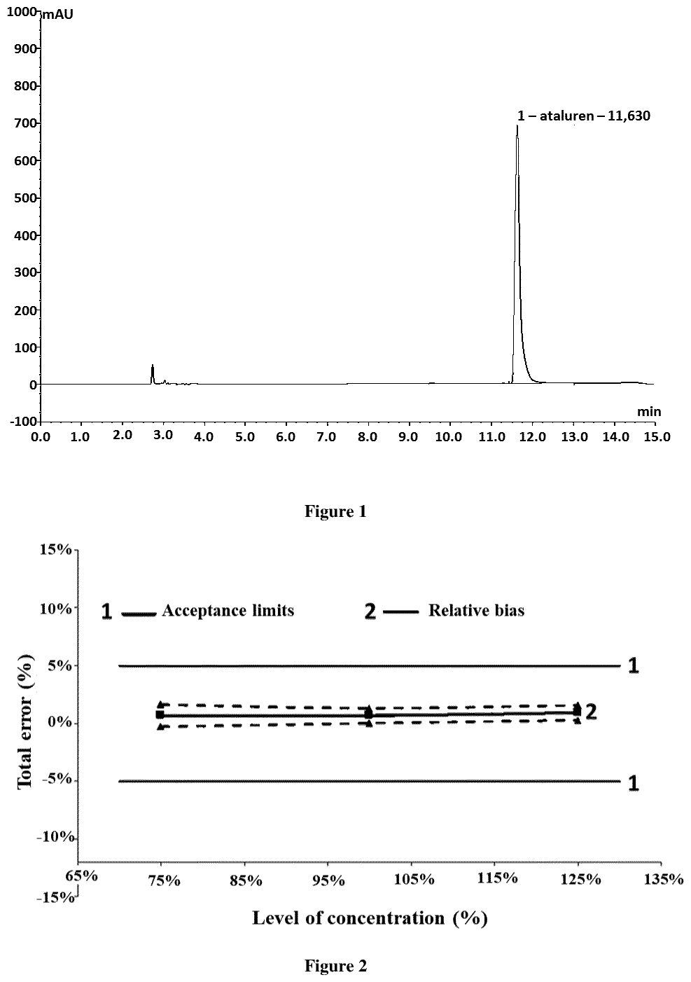

Ataluren retention time was observed to be about 11.6 min (FIG. 1). The chromatographic method used was found linear for concentration ranging from 50 to 90 μg/mL. The calculated regression parameters are given in Table 1 and are within the linearity acceptance criteria. Average regression equation was y=2.074(±0.017)x+10.250(±1.271), where x is the ataluren concentration and y is the surface area, and average determination coefficient R2 of three calibration curves was 0.9995. No matrix effect was detected. Results for intra-day precision and inter-day precision were less than 2.1% as shown in Table 2. The 95% accuracy profile was within the predefined acceptance limits (FIG. 2). The determined values of LOD and LOQ were 6.8 μg/mL and 11.1 μg/mL, respectively, calculated using slope and Y-intercept.

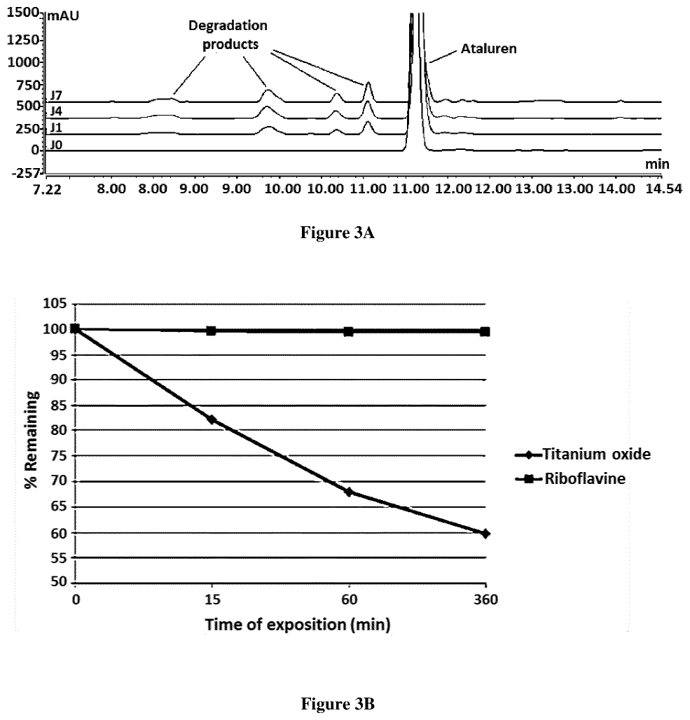

When exposed to strong acidic, basic or 0.3% H₂O₂ conditions, ataluren was not degraded after 7 days exposure (Table 3). Degradation products appeared only for 15% hydrogen peroxide exposure and were highlighted in FIG. 3a. In direct photolytic stress condition, ataluren was not degraded (Table 4). However, in presence of photocatalyst agent (titanium oxide), ataluren was rapidly degraded (FIG. 3B). This was not the case when exposing the drug to light in the presence of riboflavine. Our method is stability-indicating as it enables separation between ataluren and its degradation products without peak interferences.

Chemical Stability of Ataluren Aqueous Suspension

The chemical stability of the STAR ataluren suspension (n=6) showed a loss of the chemical stability greater than 10% at day 21 (Table 6).

Stability of Ataluren in Eyedroppers

Physical Stability

There were no detectable visual changes in color and limpidity, and no appearance of any visible particulate matter during the study period.

Chemical Stability

The ataluren 1% eye-drop oily solution stored in LDPE ophthalmic bottles at 22-25° C. demonstrated chemical stability for up to 60 days (Table 5). Ataluren retained at least 99% of its initial concentration at 60 days. Chromatograms showed no sign of degradation products throughout the study.

Sterility Assay

The sterility applicability of the method was validated according to the European Pharmacopeia assay (2.6.1). The visual microbial growth was clearly observed and comparable in presence and absence of the product to be tested. Moreover, no growth was observed for any samples analyzed with this method at day 14. Because the microbiological tests showed the absence of bacterial or fungal contamination of the preparation over time, the use of an antibacterial agent was not considered.

### DISCUSSION

Our study reports new data on the stability of ataluren in ophthalmic solutions. The use of diverse co-solvent strategies (i.e. tween 80; Cationorm®) only allowed a partial ataluren solubilization chemically stable less than 2-3 weeks unsuitable for use in eye treatments and application in humans. The use of a castor oil solution with DMSO (10%) permit to obtain a suitable 1% m/v ataluren ophthalmic solution exempt of suspended particles and stable. All parameters of the tested castor oil and DMSO formulation were in favor of a physicochemical and microbiological stability over 60 days.

Oxidation was shown critical in ataluren degradation, which is enhanced in aqueous media. The lack of water in the optimized formulation, combined to the use of pharmaceutical grade castor oil controlled for its peroxide content, could also explain an enhanced stability of ataluren in the oily solution. In addition, the presence of high level of DMSO, well-known for its antioxidant properties, may also have contributed to the absence of degradation perceived in the final formulation [21].

Simulated light experiments provided some insights of the propensity of ataluren to degrade both prior and after administration. Ataluren concentration did not decrease upon direct light exposure, pointing out that the drug may resist to light in the proposed formulation. Further, when riboflavin, a natural photosensitizing agent constituent of the eye was added, no degradation was observed, which indicates that ataluren may not degrade through photosensitized process naturally occurring in the eye [22]. Still, precaution should be taken as ataluren concentration decreased upon exposure when nanoparticles of titanium dioxide P25, a powerful photocatalyst were added [23]. This points that ataluren may degrade in the presence of strong oxidizing agent such as hydroxyl radical (OH) which was detected in the context of nuclear cataract [24,25].

The sterility assay following the European Pharmacopeia sterility monography did not reveals any microbial contamination. Moreover, the closure system eyedropper (Novelia®) which does not allow unfiltered air to penetrate the eyedropper provided a further guarantee for sterility preservation of the content more than one month of simulated use [25]. Preservative agents in ophthalmic solution can cause some damages to the ocular surface. Thus the ophthalmic solution is preservative-free and is suitable to treat aniridia impaired ocular surface.

The ataluren dose at 1% was chosen according to a preclinical study of the START therapy showing that 1% ataluren suspension gave the best efficacy in a Pax6 mouse model of aniridia [9]. However, in this study the ataluren eye-drop formulation was a suspension, which precludes its use in human eye application. For this reason, the proposed study provided the first attempt to optimized ophthalmic solution using DMSO (10%) on castor oil formulation. DMSO, known for its powerful solvating properties, is a well-known treatment for some eye diseases, and is FDA-approved in some drug products like Onyx injection, Viadur implant or Pennsaid topical gel [26-28]. Indeed, DMSO in association with dilute povidone-iodine was safely used for ophthalmic application for treatment of blepharitis in human [29], and for treatment of chronic keratitis in dogs and could enhance tissue permeation/absorption of compounds. In 11.8% of patients, mild irritation including stinging, tingling or burning at the application was experienced without serious adverse effects [24]. Castor oil is a natural derivative of the Ricinus communis plant that possesses anti-inflammatory, anti-nociceptive, antioxidant, antimicrobial properties [31-36]. Castor oil was administered safety and tolerability as a topical eye drop in mild dry eye disease, blepharitis, and contact lens discomfort, refractory meibomian gland dysfunction [33].

In view of its physicochemical stability and its preservation of sterility, this study confirms 60 days stability of 1% ataluren eye-drop without preservative when stored at 22-25° C. in LDPE ophthalmic bottles. Ataluren eye-drops aim to restore ocular surface PAX6 haploinsufficiency in congenital aniridia and this new formulation opens to further clinical studies and innovative treatment for patients.

### REFERENCES

Throughout this application, various references describe the state of the art to which this invention pertains. The disclosures of these references are hereby incorporated by reference into the present disclosure.
1. Hingorani, M.; Hanson, I.; van Heyningen, V. Aniridia. Eur. J. Hum. Genet. 2012, 20(10), 1011-1017.
2. Brémond-Gignac, D.; Gérard-Blanluet, M.; Copin, H.; Bitoun, P.; Baumann, C.; Crolla, J. A.; Benzacken, B.; Verloes, A. Three patients with hallucal polydactyly and WAGR syndrome, including discordant expression of Wilms tumor in MZ twins. Am. J. Med. Genet. A. 2005, 134(4), 422-425.
3. Gerber, S.; Alzayady, K. J.; Burglen, L.; Brémond-Gignac, D.; Marchesin, V.; Roche, O.; Rio, M.; Funalot, B.; Calmon, R.; Dun, A. et al. Recessive and Dominant De Novo ITPR1 Mutations Cause Gillespie Syndrome. Am. J. Hum. Genet. 2016, 98(5), 971-980.
4. Chrystal, P. W.; Walter, M. A. Aniridia and Axenfeld-Rieger Syndrome: Clinical presentations, molecular genetics and current/emerging therapies. Exp. Eye. Res. 2019, 189, 107815.
5. Bremond-Gignac, D. [Congenital aniridia in children]. Rev. Prat. 2019, 69(1), 67-70.
6. Lim, H. T.; Kim, D. H.; Kim, H. PAX6 aniridia syndrome: clinics, genetics, and therapeutics. Curr. Opin. Ophthalmol. 2017, 28(5), 436-447.
7. Lagali, N.; Edén, U.; Utheim, T. P., Chen, X.; Riise, R.; Dellby, A.; Fagerholm, P. In vivo morphology of the limbal palisades of vogt correlates with progressive stem cell deficiency in aniridia-related keratopathy. Invest. Ophthalmol. Vis. Sci. 2013, 54(8), 5333-5342.
8. Liu, X.; Zhang, Y.; Zhang, B.; Gao, H.; Qiu, C. Nonsense suppression induced readthrough of a novel PAX6 mutation in patient-derived cells of congenital aniridia. Mol. Genet. Genomic. Med. 2020, 3, e1198.
9. Wang, X.; Gregory-Evans, K.; Wasan, K. M.; Sivak, O.; Shan, X.; Gregory-Evans, C. Y. Efficacy of Postnatal In Vivo Nonsense Suppression Therapy in a Pax6 Mouse Model of Aniridia. Mol. Ther. Nucleic. Acids. 2017, 7, 417-428.
10. Gregory-Evans, C. Y.; Wang, X.; Wasan, K. M.; Zhao, J.; Metcalfe, A. L.; Gregory-Evans, K. Postnatal manipulation of Pax6 dosage reverses congenital tissue malformation defects. J. Clin. Invest. 2014, 124(1), 111-116.
11. PTC Therapeutics. STAR: A Phase 2, Multicenter, Randomized, Double-Masked, Placebo-Controlled Study of the Safety and Efficacy of Ataluren (PTC124) for the Treatment of Nonsense Mutation Aniridia. ClinicalTrials.gov: NCT02647359. https://clinicaltrials.gov/ct2/show/NCT02647359 #contactlocation. (accessed on 19 Oct. 2020).
12. European Medicines Agency. Translarna™ (ataluren) summary of product characteristics (2019). www.ema.europa.eu/en/documents/product-information/translarna-epar-product-information_en.pdf. (accessed on 19 Oct. 2020).
13. Peltz, S. W.; Morsy, M.; Welch, E. M.; Jacobson, A. Ataluren as an agent for therapeutic nonsense suppression. Annu. Rev. Med. 2013, 64, 407-25.
14. Roy, B.; Friesen, W. J.; Tomizawa, Y.; Leszyk, J. D.; Zhuo, J.; Johnson, B.; Dakka, J.; Trotta, C. R.; Xue, X.; Mutyam, V.; Keeling, K. M.; Mobley, J. A.; Rowe, S. M.; Bedwell, D. M.; Welch, E. M.; Jacobson, A. Ataluren stimulates ribosomal selection of near-cognate tRNAs to promote nonsense suppression. Proc. Natl. Acad. Sci. USA. 2016, 113(44), 12508-12513.
15. National Center for Biotechnology Information. PubChem Database. Ataluren, CID=11219835, https://pubchem.ncbi.nlm.nih.gov/compound/Ataluren. (accessed on 20 Oct. 2020).
16. Yeh, M. K.; Chang, L. C.; Chiou, A. H. Improving tenoxicam solubility and bioavailability by cosolvent system. AAPS PharmSciTech. 2009, 10(1), 166-171.
17. Kong R, Laskin O L, Kaushik D, Jin F, Ma J, McIntosh J, Souza M, Almstead N. Ataluren Pharmacokinetics in Healthy Japanese and Caucasian Subjects. Clin Pharmacol Drug Dev. 2019, 8(2), 172-178.
18. European Medicines Agency. ICH Topic Q1A(R2): stability testing of new drug substances and products. https://www.ema.europa.eu/en/documents/scientific-guideline/ich-q-1-r2-stability-testing-new-drug-substances-products-step-5_en.pdf. (accessed on 20 Oct. 2020).
19. EDQM (European Directorate for the Quality of Medicines & Healthcare), European Pharmacopoeia 8.0: 5.1.4 Sterility assay 04/2011:20601. EDQM Council of Europe. Strasbourg, France.
20. Food Drug and Administration. Guidance for industry: drug stability guidelines. https://www.fda.gov/media/69957/download. (accessed on 20 Oct. 2020).
21. Sanmartin-Suárez, C.; Soto-Otero, R.; Sánchez-Sellero, I.; Méndez-Álvarez, E. Antioxidant properties of dimethyl sulfoxide and its viability as a solvent in the evaluation of neuroprotective antioxidants. J. Pharmacol. Toxicol. Methods. 2011, 63(2), 209-215.
22. Challier, C.; Mártire, D. O.; García, N. A.; Criado, S. Visible light-mediated photodegradation of imidazoline drugs in the presence of Riboflavin: Possible undesired effects on imidazoline-based eye drops. J. Photochem. Photobiol. A: Chemistry. 2017, 332, 399-405.
23. Sangchay, W.; Sikonga, L.; Kooptarnond, K. Comparison of photocatalytic reaction of commercial P25 and synthetic TiO2-AgCl nanoparticles. Procedia Engineering. 2012, 32, 590-596. Nosaka, Y.; Nosaka, A. Understanding Hydroxyl Radical (OH) Generation Processes in Photocatalysis. ACS Energy Lett. 2016, 1, 2, 356-359.
24. Garner, B.; Davies, M. J.; Truscott, R. J. Formation of hydroxyl radicals in the human lens is related to the severity of nuclear cataract. Exp. Eye Res. 2000, 70(1), 81-88.
25. Roche, M.; Lannoy, D.; Bourdon, F.; Danel, C.; Labalette, P.; Berneron, C.; Simon, N.; Odou, P. Stability of frozen 1% voriconazole eye-drops in both glass and innovative containers. Eur. J. Pharm. Sci. 2020, 141, 105102.
26. Hanna, C.; Fraunfelder, F. T.; Meyer, S. M. Effects of dimethyl sulfoxide on ocular inflammation. Ann Ophthalmol. 1977, 9(1), 61-65.
27. Mckim, A.; Strub, R. Dimethyl sulfoxide USP, Ph. Eur in approved pharmaceutical products and medical devices. Pharm. Technol. 2008, 32(5), 74-85.
28. Mckim, S.; Strub, R. Advances in the regulated pharmaceutical use of dimethyl sulfoxide USP, Ph. Eur. Pharmaceut. Technol. 2016, 3, 30-35.
29. Bhalerao, H.; Koteshwara, K. B.; Chandran, S. Brinzolamide dimethyl sulfoxide In Situ Gelling Ophthalmic Solution: Formulation Optimisation and In Vitro and In Vivo Evaluation. AAPS PharmSciTech. 2020, 21(2), 69.
30. Pelletier, J.; Capriotti, K.; Stewart, K.; Barone, S.; Capriotti, J. A Novel Transdermal ophthalmic preparation for blepharitis in a dilute povidone-iodine and dimethyl sulfoxide (DMSO) system: a case series. EC Ophthalmol. 2018, 9(3), 129-134.
31. Balicki, I. Clinical study on the application of tacrolimus and DMSO in the treatment of chronic superficial keratitis in dogs. Pol. J. Vet. Sci. 2012, 15(4), 667-676.
32. Medhi, B.; Kishore, K.; Singh, U.; Seth, S. D. Comparative clinical trial of castor oil and diclofenac sodium in patients with osteoarthritis. Phyther. Res. 2009, 23, 1469-1473.
33. Oloyede, G. K. Antioxidant activities of methyl ricinoleate and ricinoleic acid dominated Ricinus communis seeds extract using lipid peroxidation and free radical scavenging methods. Res. J. Med. Plants. 2012, 6, 511-520.
34. Jena, J.; Gupta, A. K. Ricinus communis linn: a phytopharmacological review. Int. J. Pharm. Pharm. Sci. 2012, 4, 25-29.
35. Sandford, E. C.; Muntz, A.; Craig, J. P. Therapeutic potential of castor oil in managing blepharitis, meibomian gland dysfunction and dry eye. Clin. Exp. Optom. 2020, October 9.
36. CIR expert panel. Final report on the safety assessment of Ricinus Communis (Castor) Seed Oil, Hydrogenated Castor Oil, Glyceryl Ricinoleate, Glyceryl Ricinoleate SE, Ricinoleic Acid, Potassium Ricinoleate, Sodium Ricinoleate, Zinc Ricinoleate, Cetyl Ricinoleate, Ethyl Ricinoleate, Glycol Ricinoleate, Isopropyl Ricinoleate, Methyl Ricinoleate, and Octyldodecyl Ricinoleate. Int. J. Toxicol. 2006, 26, 31-77.

1. A pharmaceutical solution comprising:
i) ataluren or any one of its pharmaceutically acceptable derivatives;
ii) at least one solvent selected from the group consisting of dimethylsulfoxide (DMSO), polyethylene glycol, polysorbate, glycerol, tyloxapol, and poloxamer; and
iii) castor oil.

2. The pharmaceutical solution of claim 1, wherein the at least one solvent is dimethylsulfoxyde (DMSO).

3. The pharmaceutical solution of claim 1, wherein the ataluren or any one of its pharmaceutically acceptable derivatives is contained in the pharmaceutical solution at a concentration ranging from 0.1 mg/mL to 15 mg/mL.

4. The pharmaceutical solution of claim 1, wherein the ataluren or any one of its pharmaceutically acceptable derivatives is contained in the pharmaceutical solution at a concentration of 10 mg/mL.

5. The pharmaceutical solution of claim 1, wherein the ataluren or any one of its pharmaceutically acceptable derivatives contained in the pharmaceutical solution is fully solubilized.

6. The pharmaceutical solution of claim 1, wherein the pharmaceutical solution contains 50% to 99% castor oil.

7. The pharmaceutical solution of claim 6, wherein the pharmaceutical solution contains 90% castor oil.

8. The pharmaceutical solution of claim 1, wherein the pharmaceutical solution does not comprise a preservative agent.

9. The pharmaceutical solution of claim 1, wherein the pharmaceutical solution is a sterile solution

10. A container comprising the pharmaceutical solution according to claim 1.

11. The container of claim 10, wherein the container is an ophthalmic bottle.

12. A method for treating aniridia in a subject in need thereof comprising administering to the subject a therapeutically effective amount of the pharmaceutical solution according to claim 1.

13. A method for preparing the pharmaceutical solution according to claim 1 comprising the steps of:
a) providing ataluren or any one of its pharmaceutically acceptable derivatives in powder form;
b) mixing said ataluren or pharmaceutically acceptable derivatives thereof in powder form, with at least one solvent selected from the group consisting of dimethylsulfoxyde (DMSO), polyethylene glycol, polysorbate, glycerol, tyloxapol, and poloxamer; and
c) adding castor oil and mixing the solution obtained, thereby preparing the pharmaceutical solution.

14. The method according to claim 13, wherein the following steps are added:
d) filtering the pharmaceutical solution obtained in step c), and
e) distributing the filtered pharmaceutical solution into a container.
